(19) United States
(12) Patent Application Publication
(10) Pub. No.: US 2006/0069377 A1
(43) Pub. Date: Mar. 30, 2006

(54) Diaper ear manufacturing process

(76) Inventors: Mickey Calvert, Norcross, GA (US); Carol Erdman, West Chester, PA (US)

Correspondence Address: Mark S. Leonardo, Esq.;Brown Rudnick Berlack Israels LLP, One Financial Center, Boston, MA, 02111, US

(21) Appl. No.: 10/951,520

(22) Filed: Sep. 28, 2004

(51) Int. Cl. A61F 13/15

(52) U.S. Cl. 604/391; 604/390

(57) ABSTRACT

An absorbent article is provided including a backsheet, a topsheet and an absorbent core disposed therebetween. The diaper includes a pair of fasteners each of which may be removably attached to a body facing surface of the diaper, and a pair of closure tabs each of which may be removably attached to a body facing side of the fastener. The fasteners and/or closure tabs may be attached to the diaper by ultrasonic, adhesive, compression or thermal bonding. A method of making the diaper is also disclosed and includes the steps of folding the fastener and/or closure tab over a portion of the topsheet, and attaching the fastener and/or closure tab to a portion of the topsheet.

### BACKGROUND

1. Technical Field

The present disclosure relates generally to disposable absorbent articles, and more particularly, to the manufacturing of disposable absorbent articles having laterally extending fasteners and closure tabs.

2. Background of the Related Art

Absorbent articles such as, for example, disposable diapers, adult incontinent pads, sanitary napkins, pantiliners, incontinent garments, etc. are generally worn, in cooperation with garments and disposed against a body surface by infants or adult incontinent individuals. The absorbent article is employed to collect and absorb body fluid discharge, such as, for example, blood, menses, urine, aqueous body fluids, mucus and cellular debris. For example, the absorbent article may be disposed between the legs of an individual adjacent a crotch area. The absorbent article is positioned with a garment and drawn into engagement with a body surface of the crotch area to collect fluid discharge.

As is known, absorbent articles typically include a fluid permeable coverstock for engaging the body surface, a fluid impermeable backsheet and an absorbent core supported therebetween. The backsheet serves as a moisture barrier to prevent fluid leakage to the garment. The absorbent core usually includes a liquid retention material that faces the body surface. The absorbent core can include, for example, loosely formed cellulosic fibers, such as wood pulp, for acquiring and storing fluid discharge.

Fastening of the absorbent articles with an individual requires the use of fasteners or ear members and closure tabs that extend laterally from the body of the absorbent article. In addition, the closure tabs typically include mechanical closure material, for example, hook and/or loop material, adhesive tape or the like. For example, in typical diaper-type garments, the garment is affixed to a wearer by attaching one or more of the closure tabs that extend across the wearer's hips to hold the back and front halves of the garment to one another.

Manufacturing of these absorbent articles, however, suffers from the addition of these laterally extending fasteners and closure tabs through the assembly and manufacturing process of an absorbent article. The fasteners and/or closure tabs undesirably protrude from the body of the diaper product as it moves in the machine direction along a production line. These drawbacks can disadvantageously result in processing manufacturing malfunctions including cutting, tearing, mis-folding and otherwise misalignment of the fasteners and closure tabs. Such malfunctions result in machine down time, wasted product and unpleasant product appearance for the consumer.

It would therefore be desirable to overcome the disadvantages and drawbacks of the prior art by providing a method of manufacturing an absorbent article having fasteners and closure tabs that do not otherwise snag, tear, mis-fold or otherwise disrupt the manufacturing process, by being particularly situated or attached to the absorbent article in a manner that would prevent such manufacturing disruptions. It would also be desirable if the manufacturing of the absorbent article having fasteners and closure is made easier and more efficient.

### SUMMARY

Accordingly, an absorbent article and manufacturing process are disclosed which include an absorbent article having fasteners and/or closure tabs that are folded upon the body of the absorbent article and held in place. The advantage of this construction provides for a manufacturing process that includes less errors due to the positioning of the fasteners and closure tabs along the manufacturing travel path.

Objects and advantages of the present disclosure are set forth in part herein and in part will be obvious therefrom, or may be learned by practice of the present disclosure which is realized and attained by the instrumentalities and combinations pointed out in the appended claims for the devices and methods of the present disclosure consisting of its constituent parts, constructions, arrangements, combinations, steps and improvements herein shown and described.

In one particular embodiment, a method of making an absorbent article, which may be a diaper, is provided. The method includes providing a diaper having a backsheet, a topsheet and an absorbent core disposed therebetween and a pair of fasteners disposed along lateral portions thereof. The fastener is folded onto and adhered to the topsheet. Adherence of the fastener to the topsheet may be performed by one or more of ultrasonic bonding, adhesive bonding, compression bonding and/or thermal bonding.

In another embodiment, the method of the present disclosure provides that each fastener has a closure tab disposed along a distal portion thereof. The closure tab may be folded onto the fastener and adhered thereto by one or more of the above mentioned bonding techniques. The fastener may in turn be folded onto the topsheet and bonded thereto by one or more of the above mentioned bonding techniques.

In another embodiment, the diaper of the present disclosure includes a pair of fasteners, each of which is removably attached to a body-facing surface of the diaper, and also a pair of closure tabs, each being removably attached to a body-facing side of the fastener. The fastener may be attached to the diaper by one or more of ultrasonic bonds, adhesive bonds, compression bonds, and thermal bonds.

In yet another embodiment, the method of the present disclosure provides a diaper with a laterally disposed attachment fastener having a distally extending closure tab and mechanical closure. The attachment fastener is folded over a portion of the topsheet and the closure tab is attached to a portion of the topsheet proximal of the attachment fastener. The mechanical closure material may include hook-and-loop material, adhesive material or the like.

### BRIEF DESCRIPTION OF THE DRAWINGS

The objects and features of the present disclosure are set forth with particularity in the appended claims. The present disclosure, as to its organization and manner of operation, together with further objectives and advantages may be understood by reference to the following description, taken in connection with the accompanying drawings, in which:

FIG. 1 is a plan view of an absorbent article according to the present disclosure;

FIG. 2 is an enlarged plan view of a portion of the absorbent article shown in FIG. 1;

FIG. 3 is a partial plan view of an absorbent article according to the present disclosure;

FIG. 4 is a partial plan view of an absorbent article according to the present disclosure;

FIG. 5 is a partial plan view of an absorbent article according to the present disclosure; and

FIG. 6 is a partial plan view of an absorbent article according to the present disclosure.

### DETAILED DESCRIPTION OF EXEMPLARY EMBODIMENTS

The exemplary embodiments of the absorbent article and methods of use disclosed are discussed in terms of fluid absorbent articles, and more particularly, in terms of an absorbent article including fasteners or ear members and closure tabs that are positioned so to improve the manufacture process. The presently disclosed absorbent article avoids leakage and overflow of fluid discharge, such as, for example, blood, menses, urine, aqueous body fluids, mucus and cellular debris. It is contemplated that the absorbent article may be employed with, for example, disposable diapers, adult incontinent pads, feminine pads, sanitary napkins, or incontinent garments.

In the case of diapers and most adult incontinent products, the garment often is provided fully formed with factory-made side seams and the garment is donned by pulling it up the wearer's legs. In the case of diapers, wherein, for example, a baby lies on his or her back, a caregiver usually places the diaper between the baby's legs, pulls the front end of the diaper up between the legs and then attaches one or more closure tabs on the rear end of the diaper to the front end of the diaper, thereby forming a pant-like structure.

In the discussion that follows, the term “body-facing surface” refers to a portion of a structure that is oriented towards a body surface, and the “garment-facing surface” refers to a portion of the structure that is oriented towards a garment and is typically opposing the body-facing surface and may be referred to as such. As used herein, the term “body surface” refers to a portion of an individual's body that the absorbent article is disposed with for collecting and absorbing fluid discharge from the individual. As used herein, the term “proximal” refers to relative locations which are closer to a longitudinal centerline of the absorbent article, while the term “distal” refers to relative locations that are farther away from the longitudinal centerline of the absorbent article.

The following discussion includes a description of the absorbent article in accordance with the present disclosure. Reference will now be made in detail to the exemplary embodiments of the disclosure, which are illustrated in the accompanying Figures.

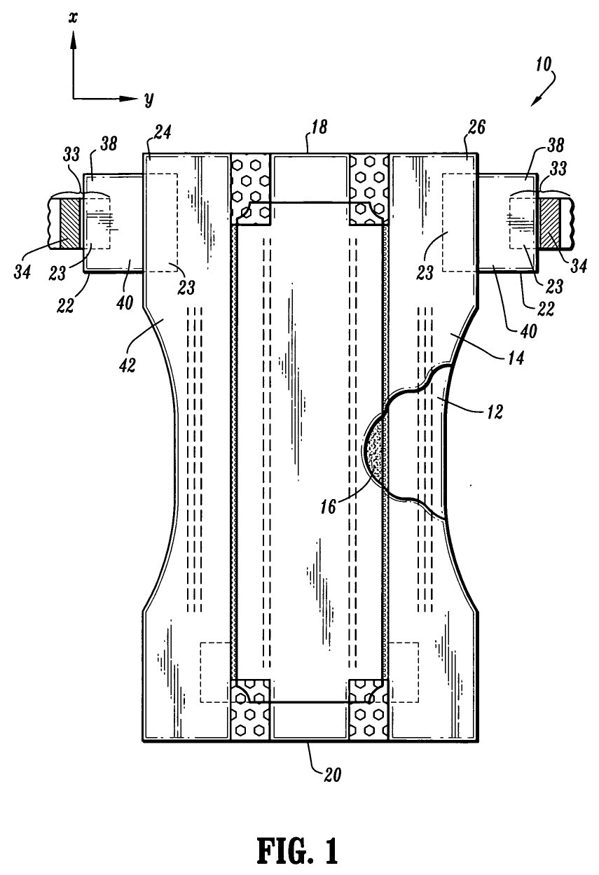

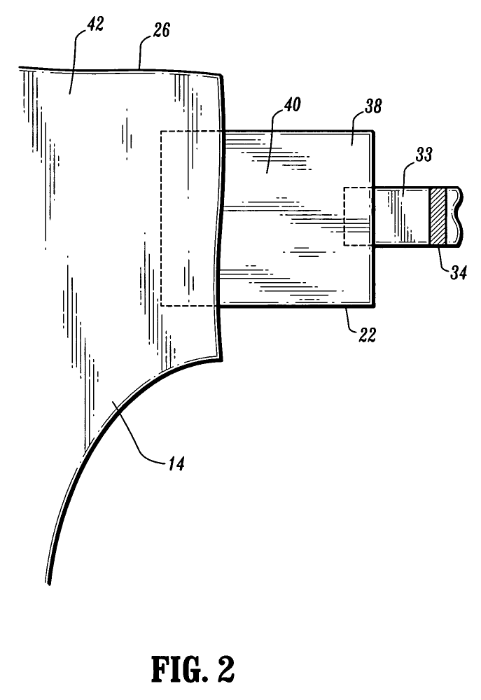

Turning now to the Figures, wherein like components are designated by like reference numerals throughout the several views. Referring to FIGS. 1-2, there is illustrated an absorbent article, constructed in accordance with the principles of the present disclosure, such as, for example diaper 10. An improved diaper 10 and method for manufacture, as compared with the prior art, is provided. Such improvement is achieved in part by providing a diaper 10 that includes a backsheet 12, a topsheet 14 and an absorbent core 16 disposed therebetween. Diaper 10 extends from a rear end 18, configured for engagement with a rear portion of a subject, to a front end 20, configured for engagement with a front portion of a subject, along a longitudinal axis x defined by diaper 10. A pair of fasteners 22 are disposed along respective lateral portions 24, 26 of diaper 10. Fastener 22 is made from partially elastic material and is adhesively bonded 23 to respective opposite portions 24, 26 of rear end 18. Fastener 22 is extensible in a direction along a transverse axis y relative to longitudinal axis x. Each of a pair of closure tabs 33 is attached along a distal portion 38 of fastener 22 by adhesive bonding 23. It is contemplated herein that fasteners 22 and closure tabs 33 may be formed with materials that are elastic, non-elastic or a combination thereof and may be attached to the diaper 10 or each other by adhesive, ultrasonic, thermal bonding or the like. Closure tab 33 may include any hook-and-loop type material, adhesive, or other type of mechanical closure material 34 that is capable of holding diaper 10 on a wearer. Closure tab 33 operates by engaging with or adhering to a corresponding surface, landing zone or object (not shown) located on the opposite end of the diaper 10, preferably front end 20.

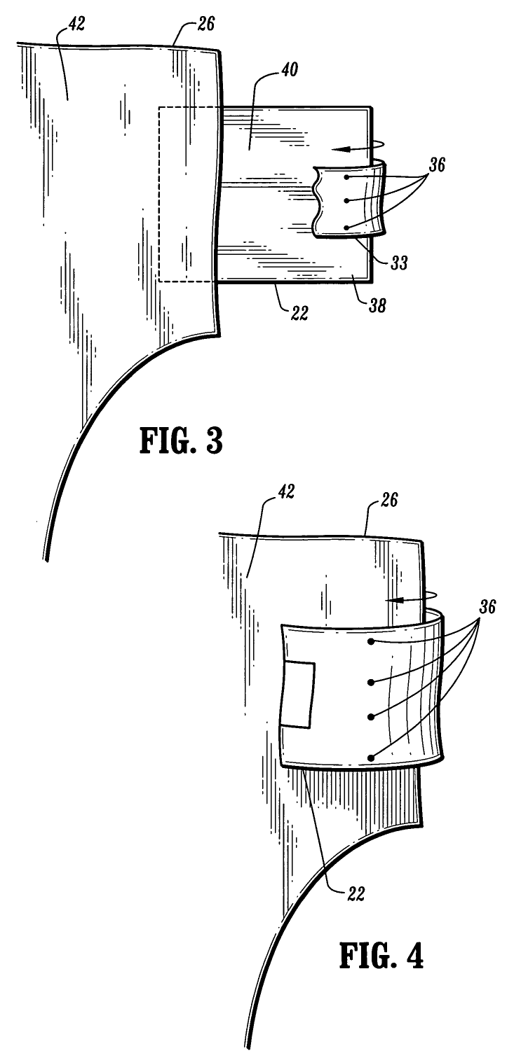

With reference to FIGS. 3 and 4, the present disclosure provides that closure tab 33 is removably attached to a body-facing surface 40 of fastener 22. More specifically, closure tab 33 is secured to fastener 22 by one or more spaced apart bonds 36. Bonds 36 may include point bonds that allow for minimum removal force by a user. The point bonds may be applied by ultrasonic, adhesive, compression, thermal bonding or other suitable means. It is contemplated herein that the bonds 36 be formed from a variety of sizes and dimensions so as to provide a variety of pull-off forces as required by the particular application. Fastener 22 is removably attached to a body-facing side 42 of diaper 10. Again, fastener 22 is secured to diaper 10 by one or more bonds 36 applied by one of the above-mentioned bonding techniques.

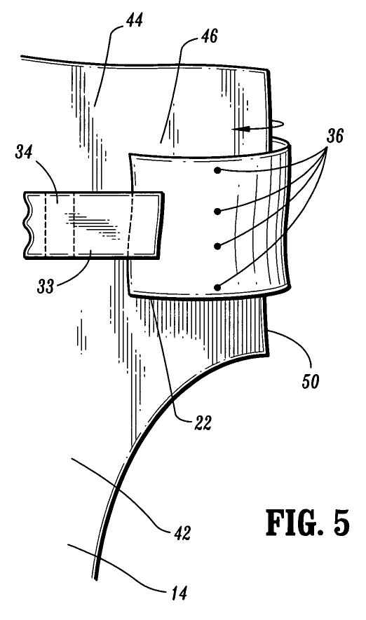

An alternate embodiment of the diaper 10 and manufacturing method, similar to that described above, is illustrated in FIG. 5. In this case, diaper 10 includes a laterally disposed attachment fastener 22 having a distally extending closure tab 33. In addition, closure tab 33 includes on its inner surface a mechanical fastener 34 (shown in phantom). According to the present embodiment, attachment fastener 22 is folded over a portion 46 of topsheet 14 proximal of diaper edge 50. Closure tab 33 is attached to a portion 44 of topsheet 14 proximal of attachment fastener 22. To facilitate attachment of closure tab 33 to topsheet 14, mechanical fastener 34 may include hook-and-loop material, adhesive, or other closure material capable of mating with partner material (not shown) or topsheet 14. Alternatively, increased attachment may be provided using ultrasonic or other previously described attachments means or bonds 36 to secure attachment fastener 22 and/or closure tabs 33 to topsheet 14.

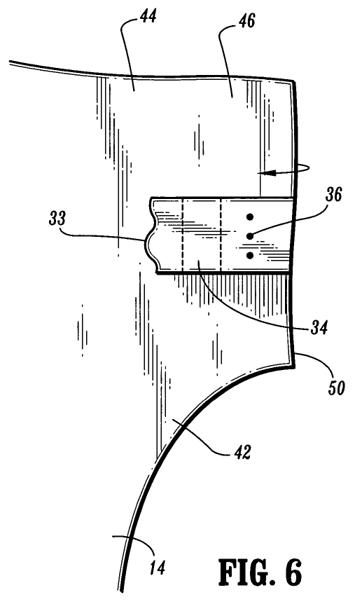

In yet an alternate embodiment of the diaper 10 and manufacturing method, similar to that described above, is illustrated in FIG. 6. A distally extending closure tab 33 is directly attached to diaper 10. Closure tab 33 includes on its inner surface a mechanical fastener 34 (shown in phantom). According to the present embodiment, closure tab 33 is folded over a portion 46 of topsheet 14 proximal of diaper edge 50. To facilitate attachment of closure tab 33 to topsheet 14, mechanical fastener 34 may include hook-and-loop material, adhesive, or other closure material capable of mating with partner material (not shown) or topsheet 14. Ultrasonic or other previously described attachments means or bonds 36 may be optionally applied to secure closure tab 33 to topsheet 14. It is contemplated herein that a distal portion 46 of topsheet 14 may also be folded over upon itself and held in place through the securing of closure tab 33 onto a more proximal portion of topsheet 14.

A method of manufacturing a diaper 10, similar to that described, in accordance with the principles of the present disclosure is provided. The process can best be understood by reference to the illustrative embodiments shown in FIGS. 2-6. FIG. 2 is an enlarged plan view of diaper 10 having fastener 22 disposed along lateral portion 26 thereof and closure tab 33 disposed along distal portion 38 of fastener 22. As illustrated in FIG. 3, closure tab 33 is folded onto a body-facing surface 40 of fastener 22. Alternatively, closure tab 33 may be folded directly upon topsheet 14 (FIG. 5). Folded closure tab 33 is then secured to fastener 22 by application of one or more attachment bonds 36. In this manner, closure tab 33 is made removably attached to fastener 22. Alternatively, attachment bonds 36 may also include one or more of adhesive, compression, thermal or other suitable bonds. Alternatively, fastener 22 (with closure tab 33 secured thereto) is folded onto the body-facing side 42, that is, topsheet 14 of diaper 10. The folded fastener 22 is lightly secured to topsheet 14 by application of one or more attachment bonds 36. In this manner, fastener 22 is removably attached to topsheet 14. Fastener 22 and diaper 10 may also be adhered together, for example, by adhesive, compressive or thermal bonding.

The present disclosure provides an improved method of manufacturing diaper 10. During the manufacturing process, fasteners 22 and/or closure tabs 33 are maneuvered so that they do not extend beyond diaper edges 50. Instead, fastener 22 and/or closure tab 33 are folded and/or attached to the diaper body during the manufacture and packing process. These attachments of the fastener 22 and closure tab 33 may be lightly secured to allow relatively easy release during usage. In addition, attachment bonds 36 used to secure fastener 22 and closure tab 33 may be small and generally minimally visible to a user (e.g., point bonds). In this manner, the present disclosure provides a more aesthetic and consumer-friendly diaper product. In addition, the user upon unpacking diaper 10 will conveniently find fasteners 22 and closure tabs 33 in the same unobtrusive position every time, that is, folded along the body of diaper 10.

It will be understood that various modifications may be made to the embodiments disclosed herein. For example, the present disclosure contemplates the use of any one or combination of embodiments discussed herein. By way of non-limiting example, the diaper 10 of the present disclosure may include folded closure tab 33 onto fastener 22 as shown in FIG. 3 or the combination of folded closure tab 33 onto fastener 22 and folded fastener 22 onto topsheet 14 as shown in FIG. 4. Therefore, the above description should not be construed as limiting, but merely as exemplification of the various embodiments. Those skilled in the art will envision other modifications within the scope and spirit of the claims appended hereto.

What is claimed is:

1. A method of making a diaper, the method comprising the steps of:
providing a diaper including a backsheet, a topsheet and an absorbent core disposed therebetween, the diaper having a fastener and a closure tab disposed along a lateral portion thereof;
folding the fastener onto the topsheet; and
adhering the fastener to the topsheet.

2. The method according to claim 1, wherein the step of adhering includes bonding the fastener to the topsheet.

3. The method according to claim 2, wherein the bonding includes point bonds selected from the group consisting of ultrasonic bonds, adhesive bonds, compression bonds and thermal bonds.

4. A method of making a diaper, the method comprising the steps of:
providing a diaper including a backsheet, a topsheet and an absorbent core disposed therebetween, the diaper having a fastener disposed along a lateral portion thereof, wherein the fastener includes a closure tab disposed along a distal portion thereof;
folding the closure tab onto the fastener; and
adhering the closure tab to the fastener.

5. The method according to claim 4, further comprising the step of folding the fastener onto the topsheet.

6. The method according to claim 5, further comprising the step of bonding the fastener to the topsheet.

7. The method according to claim 6, wherein the bonding includes point bonds selected from the group consisting of ultrasonic bonds, adhesive bonds, compression bonds, and thermal bonds.

8. The method according to claim 4, wherein the step of adhering is performed by one of ultrasonic bonding, adhesive bonding, compression bonding and thermal bonding.

9. A diaper comprising:
a backsheet, a topsheet and an absorbent core disposed therebetween;
a pair of fasteners disposed along lateral portions of the diaper, each fastener being removably attached to a body facing side of the diaper; and
a pair of closure tabs disposed along distal portions of the fastener, each closure tab being removably attached to a body facing side of the fastener.

10. The diaper according to claim 9, wherein the fastener is attached to the diaper by one of ultrasonic, adhesive, compression or thermal bonding.

11. A diaper comprising:
a backsheet, a topsheet and an absorbent core disposed therebetween;
a fastener disposed along a lateral portion of the diaper, wherein the fastener is attached to the diaper along at least two separate spaced locations; and
a closure tab disposed along a distal portion of the fastener, wherein the closure tab is attached to the fastener along at least two separate spaced locations.

12. The diaper according to claim 11, wherein at least one of the fastener attachment locations is at a proximal end of the fastener, and at least one of the closure tab attachment locations is at a proximal end of the closure tab.

13. The diaper according to claim 12, wherein at least one of the fastener attachment locations is distal of the proximal end fastener attachment location, and at least one of the closure tab attachment locations is distal of the proximal end closure tab attachment location.

14. A method of manufacturing a diaper, the method comprising the steps of:
providing a diaper having a backsheet, a topsheet and an absorbent core disposed therebetween, the diaper including a laterally disposed attachment fastener having a distally extending closure tab, the closure tab including a mechanical fastener;
folding the attachment fastener over a portion of the topsheet; and
attaching the closure tab to a portion of the topsheet proximal of the attachment fastener.
